(19) United States
(12) Patent Application Publication
(10) Pub. No.: US 2016/0178587 A1
(43) Pub. Date: Jun. 23, 2016

(54) TEMPERATURE REGULATION OF GAS DETECTOR BY CO-OPERATING DUAL HEAT SINKS AND HEAT PUMP

(71) Applicant: Crowcon Detection Instruments Limited, Abingdon, Oxfordshire (GB)

(72) Inventor: Paul Basham, Abingdon, Oxfordshire (GB)

(21) Appl. No.: 14/404,118

(22) PCT Filed: May 28, 2013
(86) PCT No.: PCT/GB2013/051395

§ 371 Date: Nov. 26, 2014

(51) Int. Cl. G01N 33/00; F28F 27/00

(52) U.S. Cl. CPC G01N 33/0027; F28F 27/00

(57) ABSTRACT

A gas detector having a temperature regulating device, the gas detector comprising a gas sensor thermally connected and embedded in a first heat sink, heat pump and a second heat sink, wherein the heat pump is thermally connected to both the first heat sink and the second heat sink, arranged such that heat energy can be transferred between the heat sinks via the heat pump to regulate the temperature of the gas sensor.

### FIELD OF INVENTION

The present invention relates to gas detectors. In particular, it relates to the temperature regulation of gas sensors.

### BACKGROUND TO THE INVENTION

It is known that the performance of gas sensing devices is dependent upon the ambient temperature and operating conditions. In particular, extreme temperatures can affect the operation of a wide-range of sensor types, including electrochemical cells, pellistors, IR sensors and luminescence-based sensors. Often sensors will have an optimal functioning temperature range and operating outside of the optimal temperature range may affect performance. Furthermore, it is known in some sensors to correct the readings to compensate for variations in operating temperature.

Another known effect that results from operating at higher temperatures is that component lifetimes are reduced. This can be attributed to various thermally-based degradation issues, including, for example, electrolyte evaporation, which can be enhanced by operating at an elevated temperature, resulting in reduced lifetime. Prolonged operation at elevated temperatures may result in evaporation of the electrolyte and subsequent sensor failure. This leads to increased costs in replacement and maintenance of the devices. Furthermore, fluctuations in operating temperatures can lead to an increased need for maintenance and calibration which will also increase the costs of operating the gas sensors.

The effects highlighted above may become more relevant dependent upon the location in which the devices are being used. For example, gas sensors are often used in the Middle or Far East, where conditions are relatively extreme in terms of temperature and humidity. Indeed, ambient temperatures can be higher than 60° C. The sensors are often placed in environments which are subject to radiation from the sun, which causes the sensors to become hotter which depending on the level of heating experienced may subsequently affect their performance, as these sensors typically have a maximum operating temperature of approximately 55° C. Equally, gas sensors are commonly used in locations like Alaska or Siberia, where conditions are relatively extreme in terms of being cold. In these conditions the performance of gas sensors may also be affected, since optimal operation of gas sensors depends on the temperature of operation being stable and within a relatively narrow range of temperatures, typically −20 to 55° C.

In addition to external sources of thermal energy, electrical components used in gas-detection devices can be a further source of heat energy that can contribute to increased temperatures. This can also lead to reduced performance, if not addressed.

In order to facilitate the working of a temperature controlled gas sensor there is provided a gas detector having a temperature regulating device, comprising a gas sensor thermally connected to a first heat sink, a heat pump and a second heat sink, the heats sinks being thermally connected to the heat pump and arranged such that heat energy can be transferred between the heat sinks via the heat pump. In use, the direction of heat transfer can be controlled by operating the heat pump, such that the gas sensor is cooled, or such that the gas sensor is heated. In this embodiment the gas detector consists of a gas sensor inside a heat sink. An advantage of the invention is that whether the heat pump is powered or not powered, the thermal mass of the heat sink will make the sensor less susceptible to temperature spikes and the deleterious effects associated with them. Positioning the gas sensor inside a heat sink enables its temperature to be controlled whilst still being exposed to the flux of analyte gas and thus operating as a sensor

Advantageously, the system uses heat sinks, which do not require externally generated power sources. Combined with being relatively easily manufactured, the use of such components means that the cost of building and running such a system is reduced. Importantly, if the gas sensor temperature can be maintained or regulated to increase or decrease depending on the temperature of the surrounding environment, the reliability of the gas sensor can be improved, as well as its lifetime. This means that maintenance costs can be reduced and replacement intervals can be reduced, thereby lowering overall costs to run the system in which the sensor is used as a component.

In accordance with an aspect of the invention, there is provided a gas detector having a temperature regulating device, the gas detector comprising a gas sensor thermally connected to a first heat sink; a heat pump and a second heat sink, wherein the heat pump is thermally connected to both the first heat sink and the second heat sink, arranged such that heat energy can be transferred between the heat sinks via the heat pump to regulate the temperature of the gas sensor. The heat sinks can be operated passively or with additional cooling such as from a fan. The gas sensor is located within the heat sink enabling its temperature to be controlled accurately.

Further aspects of the invention will be apparent from the description and the claims.

### BRIEF DESCRIPTION OF THE FIGURES

Embodiments of the invention are now described, by way of example only, with reference to the accompanying drawings in which:

FIG. 1 shows a gas detector comprising a gas sensor with a cooling system according to one aspect of the invention;

FIG. 2 shows another embodiment of a gas detector comprising a gas sensor with a cooling system, wherein part of the cooling system is thermally insulated;

FIG. 3 shows a further embodiment of a gas detector comprising a gas sensor with a cooling system;

FIG. 4 shows a further embodiment of a gas detector comprising a gas sensor with a cooling system; and

FIG. 5 shows a further embodiment of a gas detector comprising a gas sensor with a cooling system.

### Detailed description of an embodiment

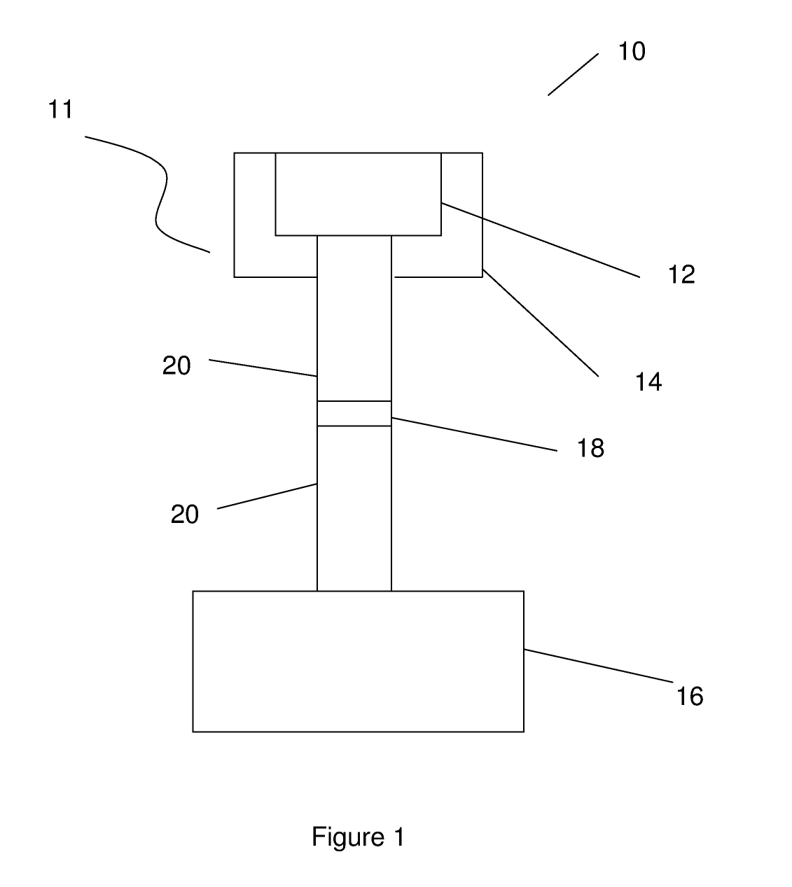

FIG. 1 is a schematic of a gas detector according to one aspect of the invention.

In FIG. 1, there is shown a gas detector 10 comprising: a gas sensor 12; a temperature regulating device 11. The temperature regulating device 11 comprises: a first heat sink 14; a second heat sink 16; a heat pump 18 which pumps heat between the first and second heat sink and thermal couplings 20. Thermal coupling 20 is in good thermal contact with the first and the second heat sink 16.

The gas sensor 12 is embedded in the first heat sink 14. Therefore, the gas sensor 12 is in thermal contact with first heat sink 14 allowing for the transfer of heat energy to and from the gas sensor to the first heat sink 14. The heat pump 18 is connected a first end to the first heat sink 14 via a thermal coupling 20. A second end of the heat pump 18 is connected to the second heat sink 16 via thermal coupling 20. Therefore the heat pump 18 can move thermal energy between the first and second heat sinks. As the first heat sink 14 is in thermal contact with the gas sensor 12, after a sufficient period of time thermal equilibrium is established between the first heat sink 14 and the gas sensor 12.

The term heat sink refers to a heat exchange component that is configured to change the temperature of a component via the transfer of thermal energy from the component to a second component. The heat sink typically has an increased surface area, volume and thermal mass, compared to the component from which thermal energy is transferred, thereby facilitating the dissipation of heat energy from the heat sink into the surrounding atmosphere.

In use, when the environment that the gas detector 10 is placed in is hotter than is required for optimum operation, and it is necessary to draw heat away from the gas sensor 12 using the temperature regulating device 11. The temperature regulating device 11 moves thermal energy from the first heat sink 14 to second heat sink 16 via the heat pump 18. As thermal energy is removed from the first heat sink 14 the heat sink cools, and because the sensor 12 is embedded in the heat sink and therefore in thermal contact the sensor is also cooled. Thus heat energy is transferred from the first heat sink 14 to the second heat sink 16. The heat energy is dissipated throughout the second heat sink 16 and excess heat energy is lost to the surrounding environment.

Alternatively, in use, when the environment that the gas detector 10 is place in is too cold for optimum operation, it is necessary to supply heat energy to the gas sensor 12 to increase the temperature of the sensor 12. The temperature regulating device 11 is configured heat energy is transferred from the second heat sink 16 to the first heat sink 14 via the heat pump 18. Thus heat energy is introduced to the first heat sink 14 and is dissipated throughout the first heat sink 14, which is in thermal equilibrium with the gas sensor 12 and thus the temperature of gas sensor 12 can be increased.

Beneficially, as the gas sensor 12 is embedded in the first heat sink 14, the temperature of the gas sensor 12 is more stable, since it is in thermal contact with the first heat sink 14 (which has a high thermal mass) and therefore less susceptible to temperature spikes. Furthermore, the embedding of the sensor 12 in the first heat sink 14 enables the temperature of the sensor to be more easily regulated, and thus kept at a constant temperature, the lifetime of the sensor may increase, and further result in lower maintenance costs. In another embodiment in which the sensor is embedded in the first heat sink 14, the first heat sink 14 is further partially thermally insulated from the surrounding environment. This allows for more efficient removal, or addition, of thermal energy at the first heat sink 14 and the gas sensor 12 and further enhances temperature regulation of the sensor 12.

The gas sensor 12 is a known, commercially available, device. Such devices typically operate at temperatures of −40 to 60° C., optimally at around 30° C. Ideally, such devices consume less than 1.6 W for certification purposes.

In an example the first and second heat sinks 14 and 16 are made from a thermally conductive material such as metal. In order to dissipate heat effectively, it is found that heat sinks constructed of a material which has a thermal conductivity of 100 W/mK or greater a particularly effective. Further it is found that heat sinks rated at 0.5 K/Watt provide the greatest effectiveness in this design of system. Heat sinks which have a lower thermal conductivity require active cooling (for example via an air fan) in order to disperse the heat and to ensure that heat can effectively be pumped between the first and second heat sinks. Preferably the heat sinks have large volumes thereby reducing thermal fluctuations and high surface areas thereby dissipating heat more efficiently and also to provide greater thermal stability of the gas sensor 12.

In an example, one or more of the heat sinks are passive components. That is to say the passive heat sinks do not require an external energy source to dissipate the heat energy introduced to the heat sink by the heat pump. It has been advantageously recognised that even in extreme environments, such as a desert environment, the use of passive heat sinks, as part of a temperature regulating device described above, can cool a gas sensor to below ambient temperature. In such a situation, the passive heat sinks are made from a relatively cheap material with a high thermal conductivity, such as extruded aluminium, though other suitable materials may be used. It is found that such materials are able to sufficiently disperse the thermal energy introduced by the heat pump and therefore maintain the temperature gradient between the first and the second passive heat sink. Thus thermal energy is directed from the first passive heat sink to the second passive heat sink enabling the first passive heat sink (and the gas sensor in thermal contact with the first heat sink) to be cooled to below ambient temperatures. In an embodiment, this is achieved by using a second passive heat sink that has a volume and/or surface area larger than the first passive heat sink. A further advantage is as the heat sink is passive, it does not require the extra cost and difficulty associated with incorporating and maintaining a power source.

In further examples, the one or more of the heat sinks are active components, requiring an external energy source in order to create a sufficient temperature gradient to dissipate heat effectively. In an example, the active heat sink has a fan associated or incorporated, in order to remove heat energy. In a further example, the active heat sink is a water-cooled heat sink.

In an example the heat pump 18 is a Peltier device which preferably forms part of the first heat sink 14. As the direction of heat transfer is determined by the flow of current through the Peltier device the reversal of electrical polarity of the device in use can cause the direction of the thermal gradient to switch and results in a change in direction of the transfer of heat energy between heat sinks 14 and 16. Therefore, the temperature regulating device 11 can either heat or cool the sensor 12 depending on the polarity of the Peltier device.

In further examples other forms of heat pump, such as a Stirling engine, are used.

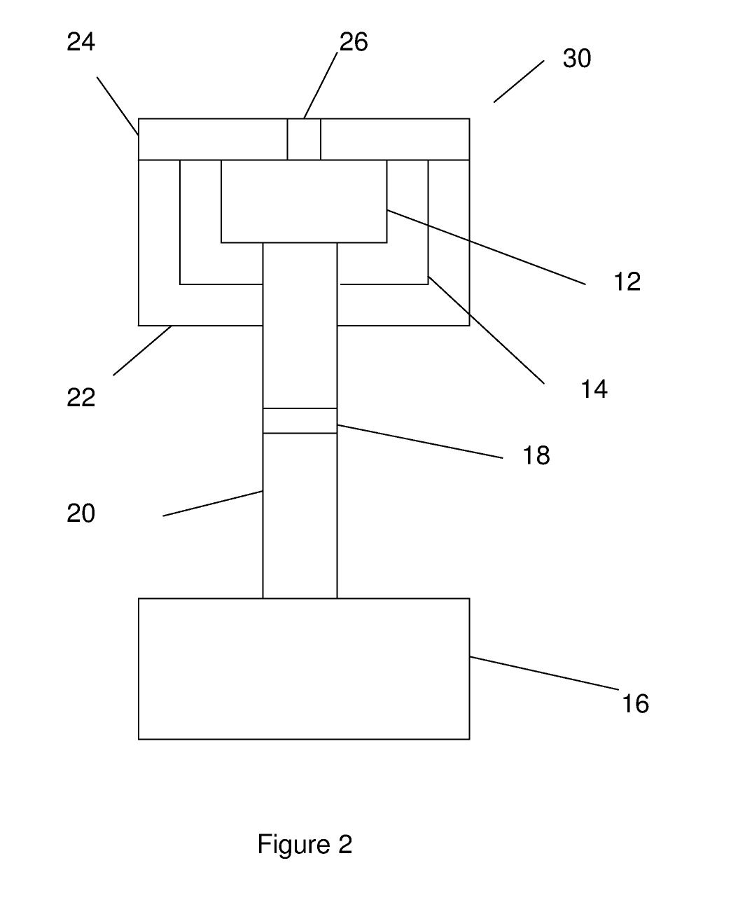

In FIG. 2 there is shown a schematic of a gas detector assembly 30 according to a further embodiment of the invention.

FIG. 2 shows a gas sensor 12 that is thermally coupled to a first heat sink 14. The first heat sink 14 is thermally coupled to part of a heat pump 18, which in turn has another part of the heat pump 18 in thermal contact with a second heat sink 16. The first heat sink is partially insulated with a thermal insulator 22. A removable chamber lid 24 is placed upon the assembly 30. There is an inlet 26 in the chamber lid 24.

The device functions as described above with reference to FIG. 1.

In use, the inlet 26 serves as an entrance for gas to reach the gas sensor 12. The chamber lid 24 provides improved thermal insulation of the gas sensor 12 and the first heat sink 14. The thermal insulation of the first heat sink 14 improves the thermal isolation of the first heat sink 14 from the surrounding environment. Heat energy will be transferred from the first heat sink 14 to the gas sensor 12 if the gas sensor 12 is cooler than the first heat sink 14. In addition, there is heat energy that is generated from the gas sensor 12 itself. Heat energy will be transferred from the surrounding environment to the first heat sink 14 and because the thermal insulation is not perfect, this process will continue until thermal equilibrium is established. When the heat pump 18 is configured such that the colder side of the heat pump 18 draws energy from the first heat sink 14, it allows a steady-state flow to be established, whereby heat energy is transferred from the first heat sink 14 to the second heat sink 16 via the heat pump 18.

Advantageously the gas sensor 12 is thermally coupled to a first heat sink 14, thereby allowing thermal energy transfer between the gas sensor 12 and the first heat sink 14. Consequently, the temperature of the gas sensor 12 is more easily controlled.

Preferably, the gas sensor 12 is embedded within the first heat sink 14 such that increased thermal contact/coupling between the gas sensor 12 and the first heat sink 14 can be established, whilst still allowing the gas sensor 12 to detect gas. The first heat sink 14 is then thermally insulated from the surrounding environment, allowing for improved thermal isolation, whilst still allowing the gas sensor 12 to detect gas, and whilst still allowing controlled heat exchange to, or from, the first heat sink 14.

Advantageously, the controlled route for thermal energy to exchange between the gas sensor 12, which is thermally connected to the partially insulated first heat sink 14, and the second heat sink 16, allows for more efficient and better controlled temperature regulation. Beneficially, due to the gas sensor 12 being in thermal contact and preferably embedded within the first heat sink 14, the gas sensor 12 is easily maintained at the temperature of the first heat sink 14. Furthermore, given that the first heat sink 14 is partially insulated, it is able to maintain its temperature more efficiently, since there will not be an excess of thermal energy loss to the surrounding environment.

Similarly, if heat energy is being drawn from the first heat sink 14 to the sensor 12, to increase the temperature of the sensor 12, as the first heat sink 14 is partially insulated from the surrounding environment, less thermal energy from the first heat sink 14 is lost to the surrounding environment. Accordingly, less thermal energy is required to heat the sensor 12.

Advantageously, in environments which are above the optimal operating temperature of the sensor 12 the insulator 22 helps maintain the sensor at a lower than ambient temperature. As heat is pumped from the first 14 to second heat sink 16 the temperature of the first heat sink and therefore sensor 12 decreases. As the first heat sink 14 is insulated by the insulator 22 the heat sink is not heated by the atmosphere. Therefore, the sensor 12 and first heat sink 14 can eventually reach a lower than ambient temperature, and preferably maintain the sensor at an optimal working temperature.

Conversely, where the device is placed in an ambient temperature is below the optimal working temperature the insulator 22 advantageously helps maintain the sensor 12 at a higher than ambient temperature. As heat is pumped into the first heat sink 14 (and therefore the sensor 12) the insulator 22 ensures that heat does not escape the heat sink 14 allowing the heat sink 14, and sensor 12, to increase in temperature.

In an example the first and second heat sinks 14 and 16 are made from a thermally conductive material such as metal. Preferably the heat sinks have large volumes (and preferably therefore a large thermal mass) and the second heat sink also has a large surface area thereby dissipating heat more efficiently and allowing greater thermal stability of the gas sensor 12.

In an example, the first heat sink 14 is thermally insulated by a thermal insulator that is made from polyurethane foam.

In an example, the first heat sink 14 is covered by a chamber lid 24 that is a thermal insulator made from polyurethane foam.

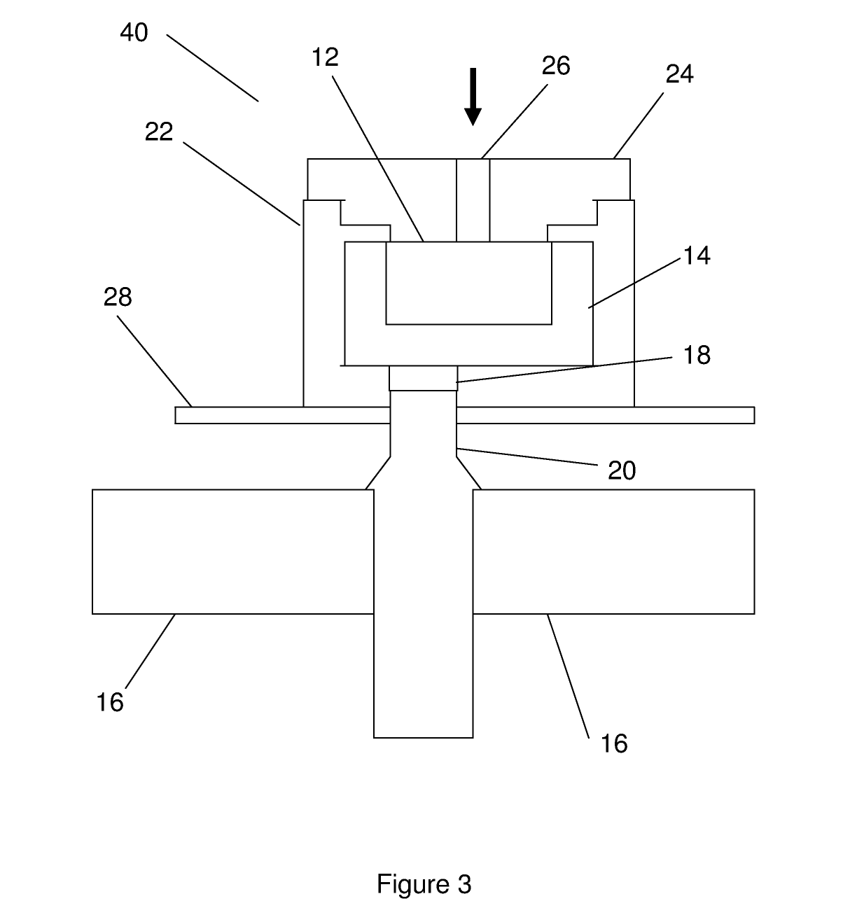

In FIG. 3 there is shown a schematic of a gas detector assembly according to one aspect of the invention.

FIG. 3 shows a gas sensor 12 in thermal contact with a first heat sink 14. The first heat sink 14 is thermally connected to a heat pump 18. The heat pump 18 is a Peltier device. The first heat sink 14 is partially thermally insulated with a thermal insulator 22 that serves as a chamber housing. The thermal insulator 22 sits on a PCB control board 28 that can be used to control the heat pump 18 and the gas sensor 12. The thermal coupling 20 between the heat pump 18 and the second heat sink 16 is a threaded metal stud.

Advantageously, the first heat sink 14 is partially thermally insulated with a thermal insulator 22, preventing significant thermal energy exchange with the surrounding environment, but furthermore it is also in thermal contact with both the gas sensor 12 and the heat pump 18, allowing conduction of thermal energy to or from the second heat sink 16. Beneficially due to the first heat sink 14 (in which the gas sensor 12 is preferably embedded) being partially thermally insulated, and thermally connected via a heat engine to a large second heat sink 16, an imbalance in the thermal equilibrium of the system will result in a net flow of thermal energy in accordance with the laws of physics. By partially thermally insulating one end of the system (i.e. the first heat sink 14) and having a second heat sink 16 with high specific heat capacity in thermal contact with it, dependent on the surrounding environment and internal generation of thermal energy, a natural temperature gradient may be created in order to control or regulate the temperature of the gas sensor 12.

Advantageously, the use of a heat pump ensures 18 that an appropriate temperature gradient can be maintained and the gas sensor 12 cooled or heated dependent on the relative temperature of the first heat sink 14.

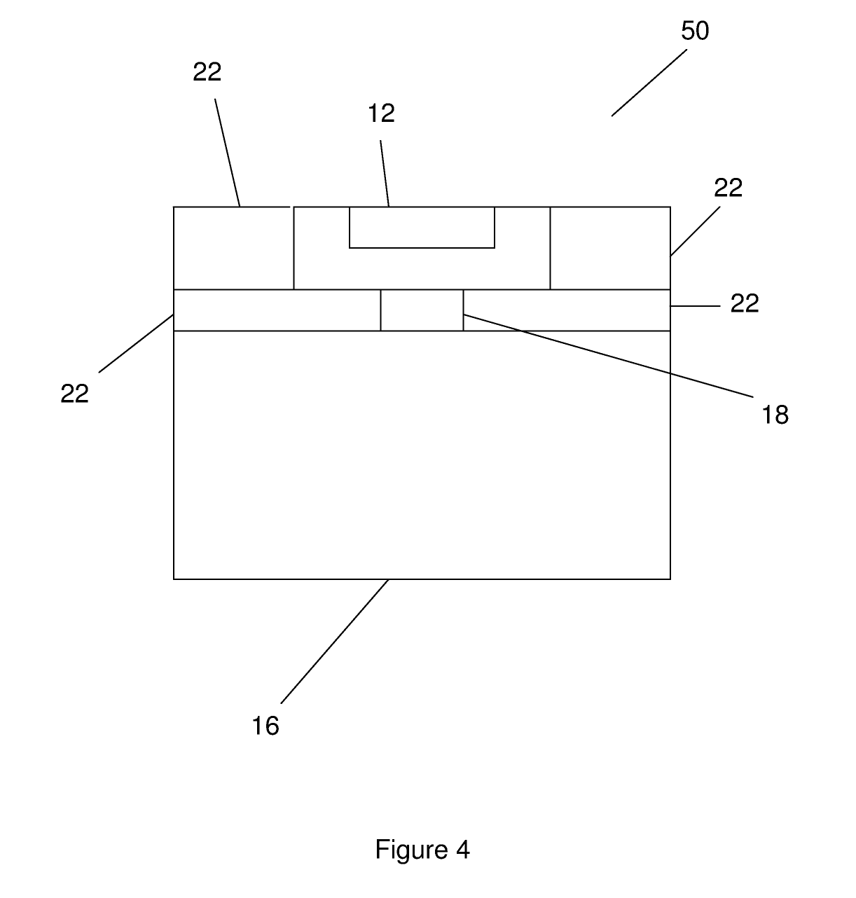

In FIG. 4 there is shown a schematic of a gas detector assembly according to one aspect of the invention.

FIG. 4 shows a gas sensor 12 in thermal contact with a first heat sink 14. The first heat sink 14 is thermally connected to part of a heat pump 18 which is in turn thermally connected by another part of the heat pump 18 to part of a second heat sink 16. The first heat sink 14 is partially thermally insulated from the surrounding environment with a thermal insulator 22. The gas detector is arranged such that the first heat sink 14 sits on top of the second heat sink 16. The components including the gas sensor 12, the first heat sink 14, the heat pump 18 and the second heat sink 16 are thermally connected directly, without the need for additional components. The first heat sink 14 and the second heat sink 16 are separated by means of a thermal insulator 22.

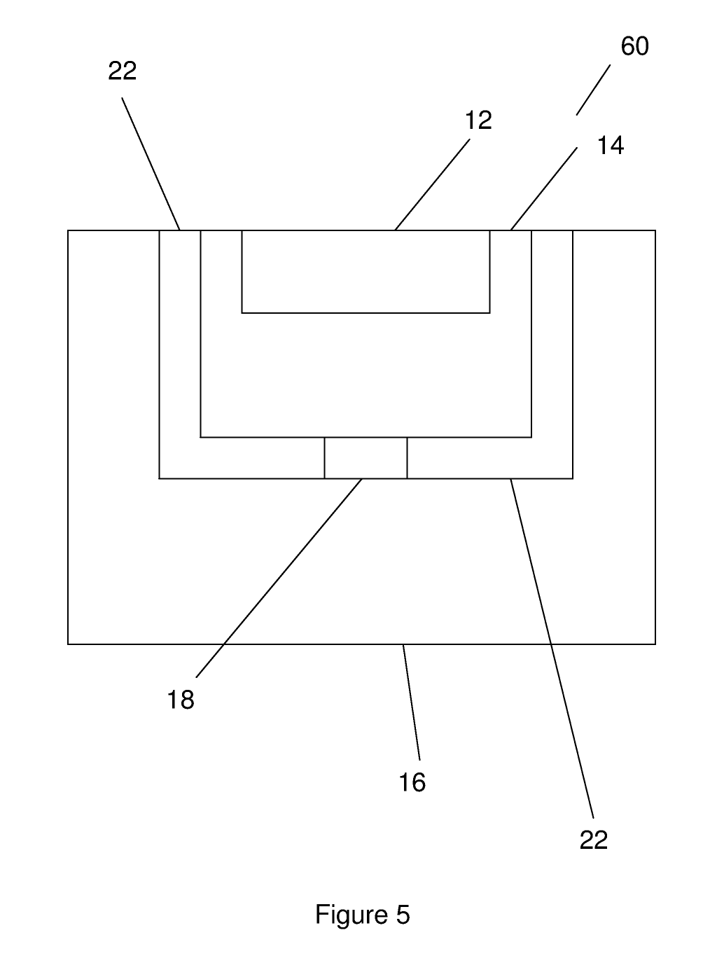

In FIG. 5 there is shown a schematic of a gas detector assembly according to one aspect of the invention.

FIG. 5 shows a gas sensor 12 thermally connected to a first heat sink 14. The first heat sink 14 is thermally connected to part of a heat pump 18. Another part of the heat pump 18 is thermally connected to a second heat sink 16. The first heat sink 14 is partially insulated from its surrounding environment by a thermal insulator 22. The gas detector is arranged such that the first heat sink 14 is encased by the second heat sink 16. The heat sinks are separated by a thermal insulator 22 and each thermally connected to different parts of a heat pump 18. The arrangement allows for a more compact distribution of components.

In further examples, which can be used in conjunction with any of the embodiments described herein, the second heat sink is extruded and is made from aluminium. Extruded components are generally easier and cheaper to make, consequently use of such components in manufacturing may reduce costs.

In a further example, electrical components associated with the gas sensing device are embedded within the second heat sink. As the heat pumped from the first to second heat sink is low compared to the overall thermal mass of the heat sink, components in heat sink will undergo a small but manageable amount of heating. By embedding the components in the heat sink the overall size of the gas sensing device may be reduced.

In further examples one or more of the heat sinks are passive components, not requiring external energy sources. This is advantageous, because the benefits of changing or maintaining the temperature are reached without having to use an external power source that adds extra installation and maintenance costs.

In further examples the heat sinks are active components that require external energy sources in order to aid the movement of thermal energy. These can be used in situations where the amount of thermal energy that must be moved exceeds the amount achievable with passive components alone.

The gas sensing device in further examples, which can be used in conjunction with any of the embodiments described herein, further comprises a thermometer and thermostat (not shown). The thermostat is configured to regulate the heat pump in order to maintain the sensor 12 at the desired working temperature.

The present invention therefore provides a heat regulating device which can cool a gas sensing device in an effective manner. As the heat pump 18 is a Peltier device there are few moving parts, and therefore requires little or no maintenance. Furthermore, the cost of manufacture of the heat regulating device 11 can be kept low.

The present invention advantageously properties because the gas sensor is within the heat sink. The heat regulating device 11 does not require a further energy source in order to dissipate the energy extracted from the relevant heat sink. Thus the heat regulating devices only requires power to heat pump thereby reducing the overall energy budget and component cost.

Advantageously, by maintaining the sensor 12 at an optimal working temperature the problems associated with extreme temperatures are mitigated.

In further examples, which can be used in conjunction with any of the embodiments herein described, the gas detector is relatively small and therefore easy to install.

1-24. (canceled)

25. A gas detector comprising:
a first heat sink;
a gas sensor themally coupled to the first heat sink;
a second heat sink; and
a heat pump thermally coupled to the first heat sink and the second heat sink and configured to transfer thermal energy between the first heat sink and the second heat sink to regulate a temperature of the gas sensor.

26. The gas detector of claim 25, wherein the first heat sink is at least partially encased by a thermal insulating material to at least partially thermally insulate the first heat sink from a surrounding environment.

27. The gas detector of claim 26, wherein the first heat sink is formed from a material having a higher thermal conductivity than the thermal insulating material.

28. The gas detector of claim 26, wherein the thermal insulating material forms a housing having a removable lid.

29. The gas detector of claim 28, wherein the removable lid includes an inlet formed therein, the inlet providing fluid communication between the surrounding environment and the gas sensor.

30. The gas detector of claim 25, wherein the gas sensor is at least partially encased by a thermal insulating material to at least partially thermally insulate the gas sensor from a surrounding environment.

31. The gas detector of claim 25, wherein the gas sensor is embedded in the first heat sink.

32. The gas detector of claim 25, wherein the heat pump is one of a Peltier device and a Stirling device.

33. The gas detector of claim 25, wherein the gas sensor is at least partially encased within the first heat sink, an active portion of the gas sensor at least partially exposed to a surrounding environment.

34. The gas detector of claim 25, wherein the first heat sink is mounted on a PCB control board for controlling at least one of the heat pump and the gas sensor.

35. The gas detector of claim 25, wherein at least one of the first heat sink and the second heat sink is thermally coupled to the heat pump by a threaded metal stud.

36. The gas detector of claim 25, wherein the heat pump is configured to generate a temperature gradient wherein heat energy is transferred from the first heat sink to the second heat sink to cause the gas sensor to be cooled to a temperature below an ambient temperature.

37. The gas detector of claim 25, wherein the heat pump is configured to generate a temperature gradient wherein heat energy is transferred from the second heat sink to the first heat sink to cause the gas sensor to be heated to a temperature above an ambient temperature.

38. The gas detector of claim 25, wherein the second heat sink has a volume greater than a volume of the first heat sink.

39. The gas detector of claim 25, wherein the first heat sink is passively cooled.

40. The gas detector of claim 25, wherein the second heat sink is passively cooled.

41. A gas detector having a temperature regulating device, the gas detector comprising:
a first heat sink at least partially encased by a housing formed from a thermal insulating material;
a gas sensor thermally coupled to the first heat sink;
a second heat sink; and
a heat pump thermally coupled to both the first heat sink and the second heat sink and configured to transfer thermal energy between the first heat sink and the second heat sink to regulate the temperature of the gas sensor.

42. The gas detector of claim 41, wherein the housing includes a removable lid, the removable lid having an inlet formed therein for providing fluid communication between the gas sensor and a surrounding environment.

43. The gas detector of claim 41, wherein the first heat sink is formed from a material having a higher thermal conductivity than the thermal insulating material.

44. The gas detector of claim 41, wherein the gas sensor is at least partially encased by the first heat sink and at least a portion of the gas sensor is in fluid communication with a surrounding environment.
